(19) United States
(12) Patent Application Publication
(10) Pub. No.: US 2007/0110611 A1
(43) Pub. Date: May 17, 2007

(54) Method of Disinfecting Items In a Vacuum Using Ozone

(75) Inventors: Alfredo J. Teran, Cape Canaveral, FL (US); Nidal A. Samad, Merritt Island, FL (US); Louis V. Mangiacapra, Mims, FL (US)

Correspondence Address: SMITH HOPEN, PA, 180 PINE AVENUE NORTH, OLDSMAR, FL, 34677, US

(73) Assignee: AJT & ASSOCIATES, INC., Cape Canaveral, FL (US)

(21) Appl. No.: 11/552,747

(22) Filed: Oct. 25, 2006

Related U.S. Application Data
(60) Provisional application No. 60/596,862, filed on Oct. 26, 2005.

(51) Int. Cl. A61L 2/18

(52) U.S. Cl. 422/28; 422/186.07

(57) ABSTRACT

A method of disinfecting or sterilizing an article, such as medical devices or tools, is provided. The items are sterilized by placing them in an enclosed chamber and evacuating the air from the vacuum Ozonated vapor is then injected into the chamber for a predetermined time allowing the ozonated vapor to contact, and destroy, the pathogens in the chamber. The ozonated vapor is injected for a predetermined time and at a predetermined pressure. Upon completion of the disinfection or sterilization cycle, or sub-cycle, the ozonated vapor is evacuated from the chamber by reestablishing a vacuum.

### CROSS REFERENCE TO RELATED APPLICATIONS

This application claims priority to currently pending U.S. Provisional Patent Application 60/596,862, filed Oct. 26, 2005.

### BACKGROUND OF THE INVENTION

Disinfection is considered to be the primary mechanism for the inactivation/destruction of pathogenic organisms present on articles to prevent the spread of diseases to downstream users and the environment. It is important that items such as medical devices and tools be properly disinfected/sterilized prior to reuse.

Ozone is produced when oxygen (O2) molecules are dissociated by an energy source into oxygen atoms and subsequently collide with an oxygen molecule to form an unstable gas, ozone (O3), which is used to disinfect wastewater. Most wastewater treatment plants generate ozone by imposing a high voltage alternating current (6 to 20 kilovolts) across a dielectric discharge gap that contains an oxygen-bearing gas. Ozone is generated onsite because it is unstable and decomposes to elemental oxygen in a short amount of time after generation. Ozone is a very strong oxidant and virucide. The mechanisms of disinfection using ozone include: direct oxidation/destruction of the cell wall with leakage of cellular constituents outside of the cell; reactions with radical by-products of ozone decomposition; damage to the constituents of the nucleic acids (purines and pyrimidines); and breakage of carbon-nitrogen bonds leading to depolymerization.

When ozone decomposes in a solvent such as water, the free radicals hydrogen peroxy (HO2) and hydroxyl (OH) that are formed have great oxidizing capacity and play an active role in the disinfection process. It is generally believed that the bacteria are destroyed because of protoplasmic oxidation resulting in cell wall disintegration (cell lysis). The effectiveness of disinfection depends on the susceptibility of the target organisms, the contact time, and the concentration of the ozone.

Advantages of using ozone over traditional sterilization techniques are numerous. For example, ozone is more effective than chlorine in destroying viruses and bacteria and in most cases the ozonation process utilizes a short contact time (approximately 10 to 30 minutes). There are no harmful residuals that need to be removed after ozonation because ozone decomposes rapidly. There is no regrowth of microorganisms after ozonation. Ozone is also generated onsite, and thus, there are fewer safety problems associated with shipping and handling.

Ozone disinfection is generally used at medium to large sized plants after at least secondary treatment. In addition to disinfection, another common use for ozone in wastewater treatment is odor control. Ozone disinfection is the least used method in the United States. Ozone treatment has the ability to achieve higher levels of disinfection than either chlorine or UV, however, the capital costs as well as maintenance expenditures have not been competitive with available alternatives. Ozone is therefore used only sparingly, primarily in special cases where alternatives are not effective. Therefore, what is needed is a cost-effective solution that is capable of using the effective sterilization power of ozone in a compact device.

### SUMMARY OF INVENTION

In one embodiment, the invention includes a method of disinfecting an article, such as medical devices or tools. The items are sterilized by placing them in an enclosed chamber and evacuating the air to form a vacuum. Ozonated vapor is then injected into the chamber for a predetermined time allowing the ozonated vapor to contact, and destroy, the pathogens in the chamber. The ozonated vapor is injected for a predetermined time and at a predetermined pressure, for example 15 psig. Upon completion, the ozonated vapor is evacuated from the chamber by reestablishing a vacuum.

In an alternative embodiment a solvent is injected into the chamber for a predetermined time prior to establishing a vacuum and introducing the ozonated vapor. The chamber is drained after a sufficient time as passed to allow the solvent to dissolve the organic matter in the chamber. An illustrative solvent is alcohol, which also displays significant disinfecting characteristics.

In one embodiment, the ozonated vapor is generated by a device comprising an ozone source communicatively coupled to an ozone conduit having a discharge at one end. A fluid reservoir is communicatively coupled with the ozone conduit such that the fluid in the reservoir is able to enter the ozone conduit as ozone passes there through. An atomizer is disposed on the discharge of the ozone conduit to convert the fluid from the ozone conduit into a vapor as the fluid and ozone pass there through. An absorption area adjacent the atomizer allows absorption of the ozone from the atomizer by the vapor.

In another embodiment, the ozonated vapor is generated by a device comprising an ozone source adapted to deliver ozone under pressure. An ozone conduit is placed in fluid communication with the ozone source. A fluid reservoir is disposed at the end of the ozone conduit opposite the ozone source such that ozone leaving the ozone conduit is forced into contact with the fluid in the reservoir forming an ozonated vapor. A vapor chamber in fluid communication with the fluid reservoir receives the ozonated vapor from the fluid reservoir.

In another embodiment, the ozonated vapor is generated by an ultrasonic fogging device. An illustrative fogging device comprises an ozone source communicatively coupled to an ozone conduit. An ultrasonic fogging device within a liquid reservoir creates a vapor which absorbs the ozone emanating from the discharge of the ozone conduit. The ozonated vapor is then directed through an ejection port into the chamber.

### BRIEF DESCRIPTION OF THE DRAWINGS

For a fuller understanding of the invention, reference should be made to the following detailed description, taken in connection with the accompanying drawings, in which:

FIG. 1 is a flowchart of the inventive sterilization method.

FIG. 2 is a diagram illustrative of one embodiment of an apparatus capable of carrying out the method of the current invention.

FIG. 3 is a diagram of a nebulizer capable of use in the inventive method.

FIG. 4 is a diagram of an alternate nebulizer capable of use in the inventive method.

FIG. 5 is a diagram of a fogging unit comprising an ultra-sonic fogger for use in the inventive method.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

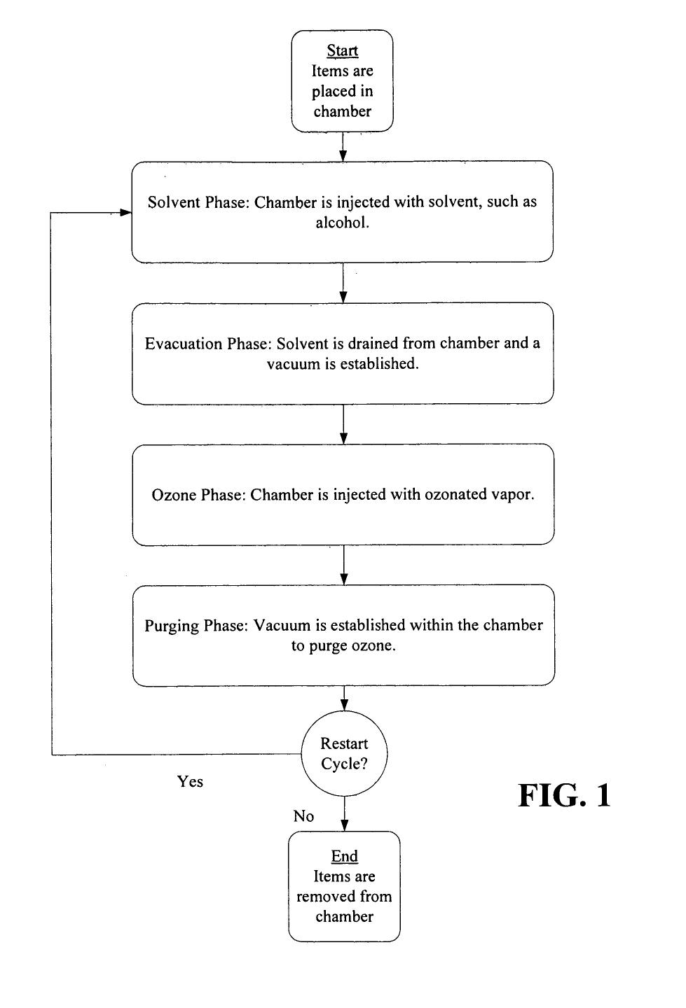

The invention includes a method for the sterilization of articles, such as medical devices, using ozone. A sterilization chamber is provided in fluid communication with an ozone source. In a preferred embodiment, the ozone source is further coupled with a device adapted to saturate a vapor with ozone prior to its introduction into the chamber. Sterilization normally occurs with the chamber sealed to provide a back pressure as the vapor enters the chamber. Sterilization, shown in FIG. 1, occurs as a result of altering the following four phases: (1) solvent phase; (2) evacuation phase; (3) ozone phase; and (4) purging phase.

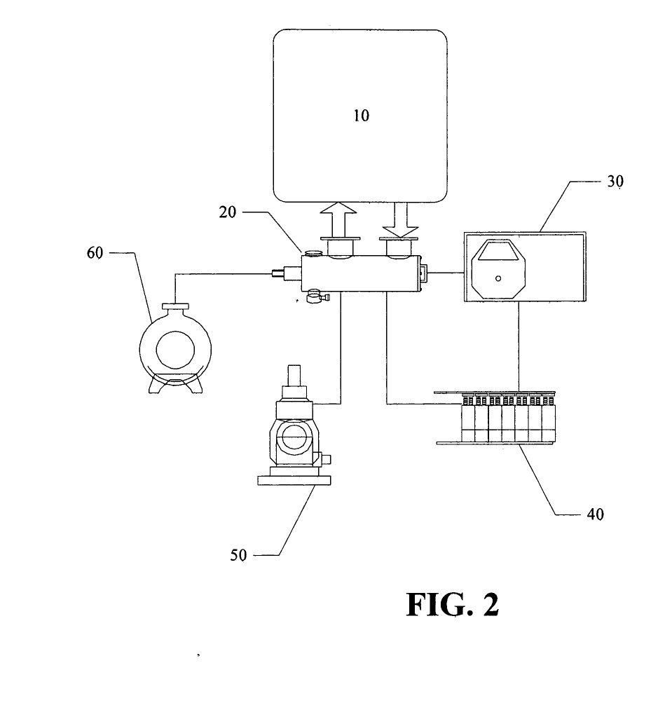

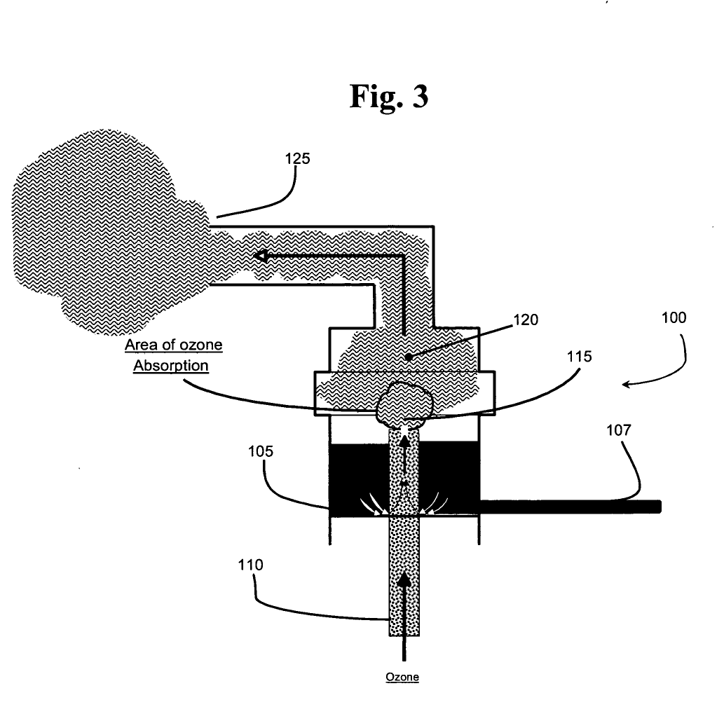

An illustrative device for implementing the inventive method, describe below, is illustrated in FIG. 2. The illustrative device comprises disinfecting/sterilization chamber 10 in fluid communication with control valve assembly 20. Control valve assembly 20 allows for establishing the vacuum within the chamber as well as providing for pressurization and introduction of the solvent and ozonated vapor. Programmable logic control unit 30 is programmed to control the sequence of the sterilization steps; including the duration of each step, concentrations of solvent and vapor and step sequence. Instrumentation 40 provides the user with information regarding internal pressure, concentrations, temperatures, cycle phase, cycle duration and the like. Ozonated vapor is provided by ozone assembly device 50. Ozone assembly device 50 can be any device adapted to produce an ozonated vapor. Illustrative devices are shown in FIGS. 3 through 5. Lastly, vacuum pump assembly 60 provides the variation in pressure necessary to establish a vacuum within the chamber to remove the ozonated vapor or solvent.

In operation, the items to be sterilized are placed within the sterilization chamber and the hatch sealed. Preferably the items have been thoroughly washed, dried and otherwise cleaned using conventional methods prior to being introduced into the chamber. It is also possible to place the instruments in a wrapper or container that is permeable to ozone and the solvent being used.

The sterilization cycle is initiated with the solvent phase. Here, a solvent is introduced into the chamber in a sufficient quantity to dissolve organic material on the surface of the articles. In one embodiment, the solvent is alcohol which exhibits significant disinfectant properties. The length of the solvent phase depends on factors such as the number of sterilization cycles being employed, type solvent, amount of solvent and the nature of the articles being sterilized.

The first evacuation phase is initiated once the solvent phase is completed. The solvent is first drained from the chamber. The solvent can be removed through a simple drain or it can be drained by establishing a positive pressure within the chamber (such as with a simple pumping mechanism). A vacuum is established once the majority of the solvent has been drained. The vacuum is established by opening the vacuum valve which is communicatively coupled to a vacuum pump. The air within the chamber is forced through the vacuum valve to a purge valve. The purge valve can be further coupled with filtration devices in situations requiring higher levels of security. The vacuum causes the remaining solvent to evaporate. The vacuum inside the disinfection chamber should be maintained for a sufficient time to ensure evaporation of the solvent (e.g. about 1 minute depending on the relative strength of the vacuum and the amount of solvent being used).

The ozone phase begins upon completion of the first evacuation phase. The chamber is injected with ozonated vapor. Ozonated vapor can be introduced into the chamber under varying parameters, such as for a predetermined time (minimum of 5 seconds) or until a desired pressure is reached within the chamber (i.e. 15 psig). The vacuum valve is closed and the vacuum pump disengaged prior to introducing the ozonated vapor into the chamber. Ozonated vapor is then injected into the chamber to reach the desired pressure and is maintained for a sufficient time for the ozone to effect sterilization of the articles in the chamber (i.e. 20 minutes). The exposure of the articles to an ozonated vapor under pressure ensures ozone penetration into all the cavities on the surface of the articles.

Additionally, the use of an ozonated vapor increases the inventions effectiveness against spore-forming pathogens, such as Anthrax. Some pathogens form protective spores in response to unfavorable conditions, such as starvation and dehydration. The resulting spore is metabolically dormant and is extremely resistant to chemical and physical attacks. The spore retains the ability to revive almost immediately when favorable conditions return to the environment. The use of ozonated vapor, due to its high humidity, degrades the she shell-like spore thereby exposing the pathogen to the ozone; thereby destroying the cell.

The final phase, the purging phase, removes the ozonated vapor from the chamber. In one embodiment, the vacuum is opened and the vacuum pump engaged. The purging phase differs from the evacuation phase in that the ozone passes through a catalyst that reverts any remaining ozone to oxygen upon removal from the chamber. The disinfected items are removed once normal pressure is established in the chamber.

One sterilization cycle, with or without the solvent step, should be used at a minimum to sterilize the items within the chamber. Additional cycles, however, can be employed and are preferred. The number of cycles can be controlled manually or by a programmable logic controller.

Any method of generating ozone can be incorporated with the invention. Ozone is measured in ppm and percent by mass or weight. Ozone can be produced with short wavelength ultraviolet radiation from a mercury vapor lamp or the application of a high voltage electrical field in a process called cold or corona discharge. The cold discharge apparatus consists of two metal plates separated by an air gap and a high dielectric strength electrical insulator such as borosilicate glass or mica. A high voltage alternating current is applied to the plates and the ozone is formed in the air gap when O₂ molecules disassociate and recombine into O₃. A faint corona may be present in the air gap, but the voltage is maintained below that which would cause punch-through of the insulator with subsequent arcing and plasma formation.

In a preferred embodiment, the ozone source is one such as that disclosed and typified in U.S. Pat. No. 5,785,864 which is incorporated herein by reference. All the pipes, conduits and surfaces of the device for implementing the inventive method are preferably constructed from non-oxidizing materials; such as PVC or stainless steel. The parts of the device that do not directly come into contact with ozone or the ozonated vapor may be constructed from other materials as desired.

Additionally, any method of saturating a vapor with ozone can be used in the invention. The following, however, represents illustrative methods of producing the ozonated vapor for use in the invention. As used herein, the term “vapor” refers to a substantially gas phase in a state of equilibrium with identical matter in a liquid or solid state below its boiling point.

### EXAMPLE I

One method of producing the ozonared vapor includes the use of a nebulizer. Nebulizer 100, as demonstrated in FIG. 3, generates ozonated water vapor 120. Water reservoir 105 is in fluid contact with ozone conduit 110. The end of ozone conduit 110 is equipped with atomizer 115. During operation, ozone passes from the ozone source through conduit 110. A small volume of water from reservoir 105 enters conduit 110 as the ozone passes through. The ozone and water combination are vaporized as it engages atomizer 115. The ozone is absorbed by the vaporized water and eventually becomes dissolved therein; thereby forming the ozonated water vapor 120. Water conduit 107 can be added to the system to replace water lost from the reservoir as vapor 120 is created. Vapor 120 then exits the device at ejection port 125 for delivery to the sterilization chamber.

### EXAMPLE II

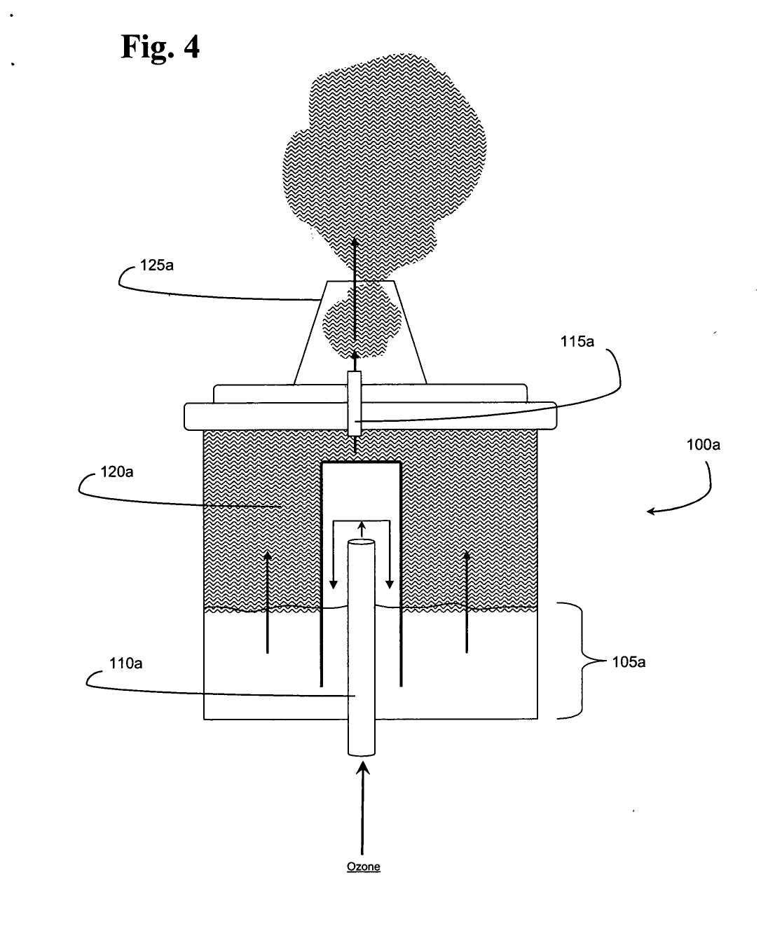

Variations of the above-described embodiment method are envisioned using any know nebulizer. For example, FIG. 4 shows alternate nebulizer 100a. Ozone leaving ozone conduit 110a enters the water contained in water reservoir 105a. Through diffusion and the pressure from conduit 110a, ozonated mist 125a forms within the apparatus where it is either dispersed through ejection port 125a. Alternatively, atomizer 115a can be adapted within the device to reduce the particle size of fog 120a.

### EXAMPLE III

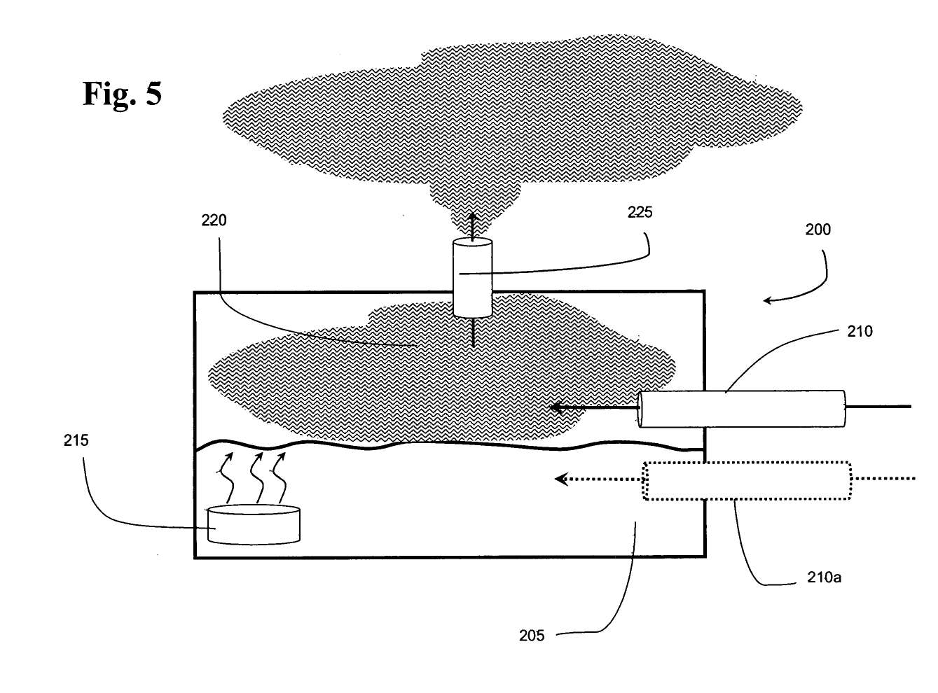

Another method of producing an ozonated vapor incorporates a misting device such as an ultrasonic fogger. As shown in FIG. 5, fogging unit 200 is a sealed container having water reservoir 205. Ultra-sonic fogger 215 is placed within reservoir 205 and creates a fog/mist comprising water vapor. Ozone enters fogging unit 200 through ozone conduit 210 and contacts the vapor in the chamber above reservoir 205; thereby forming ozonated vapor 220. It is also possible to introduce the ozone directly into the water contained in water reservoir 205 (via alternate ozone conduit 210a). Ozonated vapor 220 then exits fogging unit 200 through exit port 225. Ozonated vapor 220 is directed to the disinfection chamber for disinfection of items contained therein.

It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might fall there between. Now that the invention has been described,

What is claimed is:

1. A method of disinfecting an article, comprising the steps of:
placing the article in an enclosed chamber;
evacuating air from the enclosed chamber;
introducing an ozonated vapor into the chamber; and
evacuating the air from the enclosed chamber.

2. The method of claim 1, further comprising the steps of:
introducing a solvent into the chamber; and
draining the solvent from the chamber.

3. The method of claim 2, wherein the solvent is alcohol.

4. The method of claim 2 wherein the solvent is introduced into the chamber prior to evacuating the air from the enclosed chamber and introducing the ozonated vapor into the enclosed chamber.

5. The method of claim 1, further comprising the step of pressurizing the ozonated vapor within the enclosed chamber.

6. The method of claim 1, further comprising the step of placing the article in a permeable container within the enclosed chamber.

7. The method of claim 1 wherein the ozonated vapor is generated by a device comprising:
an ozone source;
an ozone conduit communicatively coupled to the ozone source and having a discharge at one end;
a fluid reservoir communicatively coupled with the ozone conduit such that the fluid in the reservoir is able to enter the ozone conduit as ozone passes there through;
an atomizer disposed on the discharge of the ozone conduit;
wherein said atomizer is adapted to convert the fluid from the ozone conduit into a vapor as the fluid and ozone pass there through; and
an absorption area adjacent the atomizer adapted to allow absorption of the ozone from the atomizer by the vapor.

8. The method of claim 1 wherein, the ozonated vapor is generated by a device comprising:
an ozone source adapted to deliver ozone under pressure;
an ozone conduit in fluid communication with the ozone source;
a fluid reservoir disposed at the end of the ozone conduit opposite the ozone source such that ozone leaving the ozone conduit is forced into contact with the fluid in the reservoir forming an ozonated vapor;
a vapor chamber in fluid communication with the fluid reservoir adapted to receive the ozonated vapor from the fluid reservoir; and
a vapor ejection port.

9. The method of claim 8, wherein the ozonated vapor is generated by a device further comprising an atomizer disposed between the vapor chamber and the vapor ejection port adapted to reduce the size of the vapor particles passing there through.

10. The method of claim 8, wherein ozone conduit extends to a point above the level of the fluid in the reservoir.

11. The method of claim 8, wherein the ozonated vapor is generated by a device further comprising an ozone chamber adapted to provide fluid communication from the ozone conduit to the fluid reservoir and prevent fluid communication between the ozone conduit and the vapor chamber.

12. The method of claim 1 wherein the ozonated vapor is generated by a device comprising:
an ozone source;
an ozone conduit communicatively coupled to the ozone source and having a discharge at one end;
a liquid reservoir adjacent the ozone conduit; and
a ultrasonic fogging device disposed within the liquid reservoir;
whereby ozone from the ozone conduit is absorbed by the vapor created by the ultrasonic fogging device in the liquid reservoir

13. A method of disinfecting an article, comprising the steps of:
placing the article in an enclosed chamber;
introducing a solvent into the chamber;
evacuating air from the enclosed chamber;
introducing an ozonated vapor into the chamber; and
evacuating the air from the enclosed chamber.
